(19) United States
(12) Patent Application Publication
(10) Pub. No.: US 2026/0207633 A1
(43) Pub. Date: Jul. 23, 2026

(54) COMPOSITION COMPRISING POLAR LIPIDS AND METHOD OF MAKING SAME

(71) Applicant: VAXA TECHNOLOGIES LTD, Rosh Pinna (IL)

(72) Inventors: Ohad BASHAN, Sde Varburg (IL); Isaac BERZIN, Jerusalem (IL)

(73) Assignee: VAXA TECHNOLOGIES LTD, Rosh Pinna (IL)

(21) Appl. No.: 19/567,969

(22) Filed: Mar. 16, 2026

Related U.S. Application Data
(63) Continuation of application No. 17/621,614, filed on Dec. 21, 2021.

(51) Int. Cl. A61K 31/00; A61K 47/12; A61P 25/20

(52) U.S. Cl. CPC A61K 31/658; A61K 47/12; A61P 25/20

(57) ABSTRACT

A composition for treating insomnia and related disorders is disclosed. The composition may include: (i) at least one polar lipid comprising at least one poly-unsaturated fatty acid (PUFA) moiety and (ii) Cannabidiol (CBD) or a derivative thereof.

### CROSS-REFERENCE TO RELATED APPLICATIONS

This application is a continuation application of U.S. patent application Ser. No. 17/621,614, filed on Dec. 21, 2021, which is a U.S. National Phase Application of PCT International Patent Application No. PCT/IL2020/050663, international filing date Jun. 16, 2020, which claimed the benefit of U.S. Provisional Patent Application No. 62/862,708, filed Jun. 18, 2019, which is hereby incorporated by reference in its entirety.

### FIELD OF THE INVENTION

The present invention relates to composition comprising lipids. More particularly, the present invention relates to composition comprising polar lipids and method of making same.

### BACKGROUND OF THE INVENTION

As the primary homeostatic regulator of human physiology, the human endocannabinoid system (ECS) plays a major role in the sleep-wake cycle and other circadian processes. The endocannabinoid system comprises cannabinoid receptors, their endogenous ligands, the endocannabinoids, and their biosynthetic and degradation enzymes.

How humans fall asleep, stay asleep, wake up, and remain awake is part of an internal biological process regulated by the circadian rhythms and the endocannabinoid system. The circadian rhythms govern a diverse array of actions in the body, including hormone production, heart rate, metabolism, and when to go to sleep and wake up.

Evidence of a strong relationship between endocannabinoid system and the circadian rhythms was observed in the sleep-wake cycle fluctuations of anandamide and 2-AG (the brain's own marijuana-like molecules), along with the metabolic enzymes that create and break down these endogenous cannabinoid compounds.

Anandamide is present in the brain at higher levels at night and works with the endogenous neurotransmitters oleamide and adenosine to generate sleep. Conversely, 2AG is higher during the day, suggesting that it is involved in promoting wakefulness.

The highly complex sleep-wake cycle is driven by a variety of neurochemicals and molecular pathways. Both anandamide and 2AG activate CB1 cannabinoid receptors that are concentrated in the central nervous system, including parts of the brain associated with regulating sleep.

CB1 receptors modulate neurotransmitter release in a manner that dials back excessive neuronal activity, thereby reducing anxiety, pain, and inflammation. CB1 receptor expression is thus a key factor in modulating sleep homeostasis. This is not the case, however, with respect to the CB2, the cannabinoid receptor located primarily in immune cells, the peripheral nervous system, and metabolic tissue. Whereas CB 1 receptor expression reflects cyclical circadian rhythms, no such fluctuations have been described for the CB2 receptor.

Cannabidiol (CBD) is one of many cannabinoid compounds found in cannabis. It does not appear to alter consciousness or trigger a “high.” A recent surge in scientific publications has found preclinical and clinical evidence documenting value for CBD in some neuropsychiatric disorders, including epilepsy, anxiety, and schizophrenia. Evidence points toward a calming effect for CBD in the central nervous system. Interest in CBD as a treatment of a wide range of disorders has exploded, yet few clinical studies of CBD exist in the psychiatric literature.

Administration of CBD has been shown to have differential effects on sleep based on dose. In a study among individuals with insomnia, results suggested that administration of high-dose (160 mg/day) of CBD increased total sleep time and decreased the frequency of arousals during the night while low-dose CBD has been associated with increased wakefulness.

Nevertheless, the higher doses of CBD that studies suggest are therapeutic for anxiety and insomnia, may also increase mental sedation In addition, the current retail cost of CBD could make the use high-dose administration of CBD cost prohibitive.

### SUMMARY OF THE INVENTION

Some aspects of the present invention may be directed to a composition comprising: (i) at least one polar lipid comprising at least one poly-unsaturated fatty acid (PUFA) moiety and (ii) Cannabidiol (CBD) or a derivative thereof.

In some embodiments, the at least one PUFA moiety is selected from moieties of omega-3, omega-6, omega-7, omega-9, conjugated unsaturated fatty acids and any combinations thereof.

In other embodiments, the at least one PUFA moiety is an omega-3 fatty acid moiety selected from Hexadecatrienoic acid (HTA), α-Linolenic acid (ALA), Stearidonic acid (SDA), Eicosatrienoic acid (ETE), Eicosatetraenoic acid (ETA), Eicosapentaenoic acid (EPA), Heneicosapentaenoic acid (HPA), Docosapentaenoic acid (DPA) Clupanodonic acid, Docosahexaenoic acid (DHA), Tetracosapentaenoic acid, Tetracosahexaenoic acid (Nisinic acid) and any combinations thereof.

In some embodiments, the at least one PUFA moiety is an omega-6 fatty acid moiety selected from: Linoleic acid (LA), Gamma-linolenic acid (GLA), Eicosadienoic acid, Dihomo-gamma-linolenic acid (DGLA), Arachidonic acid (AA), Docosadienoic acid, Adrenic acid (AdA), Docosapentaenoic acid (Osbond acid), Tetracosatetraenoic acid, Tetracosapentaenoic acid, and any combinations thereof.

In some embodiments, the at least one PUFA moiety is an omega-9 fatty acid moiety selected from: Oleic acid, Eicosenoic acid, Mead acid, Erucic acid, Nervonic acid, and any combinations thereof.

In some embodiments, the at least one PUFA moiety is an omega-7 fatty acid moiety selected from: palmitoleic acid, Vaccenic acid, Rumenic acid, Paullinic acid and any combinations thereof.

In some embodiments, said at least one PUFA moiety is selected from moieties of: Rumenic acid, α-Calendic acid, β-Calendic acid, Jacaric acid, α-Eleostearic acid, β-Eleostearic acid, Catalpic acid, Punicic acid, Rumelenic acid, α-Parinaric acid, β-Parinaric acid, Bosseopentaenoic acid, Pinolenic acid, Podocarpic acid and any combinations thereof.

In some embodiments, the at least one PUFA moiety is selected from DHA moiety, EPA moiety and any combinations thereof.

In some embodiments, the at least one polar lipid comprising at least one PUFA moiety is extracted from a natural source. In some embodiments, said at least one polar lipid comprising at least one PUFA moiety is extracted from a marine source. In some embodiments, the at least one polar lipid comprising at least one PUFA moiety is extracted from an algae source.

In some embodiments, the CBD is extracted from a plant source. In other embodiments, said CBD is synthetic or semi-synthetic.

In some embodiments, the at least one polar lipid comprising at least one PUFA moiety forms at least 20% wt of the composition. In other embodiments, the CBD forms at least 10% wt of the composition. In further embodiments, the ratio between the at least one polar lipid and CBD is 1:1.

In some embodiments, the EPA is at least 20% of said composition. In other embodiments, said CBD is at least 10% of said composition.

In some embodiments, a composition of the invention is in the form of an oily solution. In some embodiments, a composition of the invention is in the form of an oil-in-water solution. In some embodiments, a composition of the invention is in the form of a dry powder.

The present invention may also relate to compositions in admixture with pharmaceutically acceptable auxiliaries, and optionally other therapeutic agents. The auxiliaries may be “acceptable” in the sense of being compatible with the other ingredients of the composition and not deleterious to the recipients thereof.

Pharmaceutical compositions may include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods may include the step of bringing in association compounds used in the invention or combinations thereof with any auxiliary agent. The auxiliary agent(s), also named accessory ingredient(s), may include those conventional in the art, such as carriers, fillers, binders, diluents, disintegrants, lubricants, colorants, flavouring agents, anti-oxidants, and wetting agents.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

Embodiments of the invention may further include a pharmaceutical composition, as hereinbefore described, in combination with packaging material, including instructions for the use of the composition for a use as hereinbefore described.

For parenteral administration, suitable compositions may include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays may be contemplated. Compositions or formulations suitable for pulmonary administration e.g. by nasal inhalation include fine dusts or mists which may be generated by means of metered dose pressurized aerosols, nebulisers or insufflators.

The exact dose and regimen of administration of the composition may necessarily be dependent upon the therapeutic or nutritional effect to be achieved and may vary with the particular formula, the route of administration, and the age and condition of the individual subject to whom the composition is to be administered.

Embodiments, of the invention may further provide a composition comprising at least one polar lipid comprising at least one poly-unsaturated fatty acid (PUFA) moiety and CBD or a derivative thereof, for use in the treatment of a disease or disorder selected from sleep disorder, insomnia, depression, anxiety, attention-deficit disorder, weight disorder (obesity, anorexia) and any combinations thereof.

Embodiments, of the invention may further provide a method of treating a disease or disorder selected from sleep disorder, insomnia, depression, anxiety, attention-deficit disorder, weight disorder (obesity, anorexia) and any combinations thereof in a subject in need thereof; said method comprising administration of a composition comprising at least one polar lipid comprising at least one poly-unsaturated fatty acid (PUFA) moiety and CBD or a derivative thereof.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Some aspects of the present invention may be directed to a composition for treating insomnia and related disorders. In some embodiments, the composition may treat other neurological or hormonal conditions, for example, depression, anxiety, attention-deficit disorder, weight disorder (obesity, anorexia) and the like. Such a composition may include: (i) at least one polar lipid comprising at least one poly-unsaturated fatty acid (PUFA) moiety and (ii) Cannabidiol (CBD) or a derivative thereof.

As used herein, to a “polar lipid comprising at least one PUFA moiety” may encompass a lipid having a glycerol or a sphingosine backbone substituted at least one moiety of poly-unsaturated fatty acids (which may be the same or different) and at least one polar group being either a phosphatidyl moiety (such as for example phosphatidylcholine, phosphatidylethanolamine phosphatidylserine, phosphatidylinositols, phosphatidic acids and so forth) or a monosaccharide or oligosaccharide moiety.

The PUFA moiety may refer to any carboxylic acid moiety having an aliphatic chain of between about 4 to 28 carbon atoms and at least one double bond between at least two carbon atoms. Said at least one double bond can be in a cis, trans, E or Z configuration.

In some embodiments, the polar lipid source is plant source. In some embodiments, the plant source is microalgae. In some embodiments, the microalgae is selected from Nannochloropsis, Isochrysis, Chlorella and any combinations thereof.

In some embodiments, the at least one PUFA moiety may be selected from moieties of omega-3, omega-6, omega-7, omega-9, conjugated unsaturated fatty acids and any combinations thereof.

In other embodiments, the at least one PUFA moiety is an omega-3 fatty acid moiety selected from Hexadecatrienoic acid (HTA), α-Linolenic acid (ALA), Stearidonic acid (SDA), Eicosatrienoic acid (ETE), Eicosatetraenoic acid (ETA), Eicosapentaenoic acid (EPA), Heneicosapentaenoic acid (HPA), Docosapentaenoic acid (DPA) Clupanodonic acid, Docosahexaenoic acid (DHA), Tetracosapentaenoic acid, Tetracosahexaenoic acid (Nisinic acid) and any combinations thereof.

In some embodiments, the at least one PUFA moiety is an omega-6 fatty acid moiety selected from: Linoleic acid (LA), Gamma-linolenic acid (GLA), Eicosadienoic acid, Dihomo-gamma-linolenic acid (DGLA), Arachidonic acid (AA), Docosadienoic acid, Adrenic acid (AdA), Docosapentaenoic acid (Osbond acid), Tetracosatetraenoic acid, Tetracosapentaenoic acid, and any combinations thereof.

In other embodiments, the at least one PUFA moiety is an omega-9 fatty acid moiety selected from: Oleic acid, Eicosenoic acid, Mead acid, Erucic acid, Nervonic acid, and any combinations thereof.

In other embodiments, the at least one PUFA moiety is an omega-7 fatty acid moiety selected from: palmitoleic acid, Vaccenic acid, Rumenic acid, Paullinic acid and any combinations thereof.

In some embodiments, said at least one PUFA moiety is selected from moieties of: Rumenic acid, α-Calendic acid, β-Calendic acid, Jacaric acid, α-Eleostearic acid, β-Eleostearic acid, Catalpic acid, Punicic acid, Rumelenic acid, α-Parinaric acid, β-Parinaric acid, Bosseopentaenoic acid, Pinolenic acid, Podocarpic acid and any combinations thereof.

In some embodiments, the at least one PUFA moiety is selected from DHA moiety, EPA moiety and any combinations thereof.

In some embodiments, the at least one polar lipid comprising at least one PUFA moiety is extracted from a natural source. In some embodiments, said at least one polar lipid comprising at least one PUFA moiety is extracted from a marine source. In some embodiments, the at least one polar lipid comprising at least one PUFA moiety is extracted from an algae source.

When referring to CBD it should be understood to encompass a compound of formula (1), including any enantiomer, diastereomer, or derivative thereof. Such derivatives include, but are not limited to changes in the positions of substituents on the cyclohexene and/or phenyl rings of the CBD molecule, different lengths of hydrocarbon chains (saturated or unsaturated) on the phenyl ring and/or the cyclohexene ring of the CBD molecule, addition of substituents or removal of substituents from the phenyl ring and/or the cyclohexene ring of the CBD molecule and any combinations thereof.

(1)

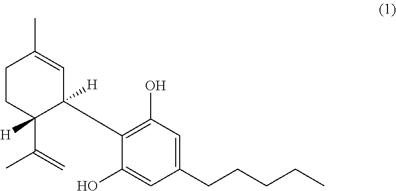 [SMILES: C=C(C)[C@@H]1CCC(C)=C[C@H]1c1c(O)cc(CCCCC)cc1O]

In some embodiments, the CBD is extracted from a plant source. In other embodiments, said CBD is synthetic or semi-synthetic.

In some embodiments, the at least one polar lipid comprising at least one PUFA moiety forms at least 20% wt of the composition. In other embodiments, the CBD forms at least 10% wt of the composition. In further embodiments, the ratio between the at least one polar lipid and CBD is 1:1.

In some embodiments, the EPA is at least 20% of said composition. In other embodiments, said CBD is at least 10% of said composition.

In some embodiments, a composition of the invention is in the form of an oily solution. In some embodiments, a composition of the invention is in the form of an oil-in-water solution. In some embodiments, a composition of the invention is in the form of a dry powder.

The present invention may also relate to compositions in admixture with pharmaceutically acceptable auxiliaries, and optionally other therapeutic agents. The auxiliaries may be “acceptable” in the sense of being compatible with the other ingredients of the composition and not deleterious to the recipients thereof.

Pharmaceutical compositions may include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods may include the step of bringing in association compounds used in the invention or combinations thereof with any auxiliary agent. The auxiliary agent(s), also named accessory ingredient(s), may include those conventional in the art, such as carriers, fillers, binders, diluents, disintegrants, lubricants, colorants, flavouring agents, anti-oxidants, and wetting agents.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

Embodiments of the invention may further include a pharmaceutical composition, as hereinbefore described, in combination with packaging material, including instructions for the use of the composition for a use as hereinbefore described.

For parenteral administration, suitable compositions may include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays may be contemplated. Compositions or formulations suitable for pulmonary administration e.g. by nasal inhalation include fine dusts or mists which may be generated by means of metered dose pressurized aerosols, nebulisers or insufflators.

The exact dose and regimen of administration of the composition may necessarily be dependent upon the therapeutic or nutritional effect to be achieved and may vary with the particular formula, the route of administration, and the age and condition of the individual subject to whom the composition is to be administered.

Embodiments, of the invention may further provide a composition comprising at least one polar lipid comprising at least one poly-unsaturated fatty acid (PUFA) moiety and CBD or a derivative thereof, for use in the treatment of a disease or disorder selected from sleep disorder, insomnia, depression, anxiety, attention-deficit disorder, weight disorder (obesity, anorexia) and any combinations thereof.

Embodiments, of the invention may further provide a method of treating a disease or disorder selected from sleep disorder, insomnia, depression, anxiety, attention-deficit disorder, weight disorder (obesity, anorexia) and any combinations thereof in a subject in need thereof; said method comprising administration of a composition comprising at least one polar lipid comprising at least one poly-unsaturated fatty acid (PUFA) moiety and CBD or a derivative thereof.

Some experiment showing the effect of the combination of CBD and four different omega-3 compounds (Fish Oil (Non-polar DHA+EPA), Fish oil (Non-polar EPA), Schizochytrium Oil (Non-Polar DHA), Nannochloropsis Oil (polar EPA)) were conducted. Daily dosage of omega 3 (DHA and/or EPA) was 500 ml.

Materials: CBD: Fish oil (non-polar DHA+EPA), Schizochytrium Oil (non-polar DHA), Nannochloropsis Oil (polar-EPA), Fish oil (non-polar EPA).

Experimental Design: Sleep quality was the measured in this study, as an efficacy indication. The experiment included 6 groups (3 participants per group) 8 women and 10 men, with similar Pittsburgh Sleep Quality Index (PSQI) rating (11-13), indicating poor sleeping quality. The average age for the participants was 37.2. All 18 participants completed sleep assessments at the onset of CBD treatment and at the monthly follow-ups.

All participants were given CBD and/or Omega 3 every evening, after dinner, per table 1 below. Sleep quality was tracked on a monthly basis, for a period of up to 4 months, using the PSQI. This Index is a self-report measure that assesses the quality of sleep during a 1-month period. It consists of 19 items that have been found to be reliable and valid in the assessment of a range of sleep-related problems. The PSQI measures several different aspects of sleep, offering seven component scores and one composite score. The component scores consist of subjective sleep quality, sleep latency (i.e., how long it takes to fall asleep), sleep duration, habitual sleep efficiency (i.e., the percentage of time in bed that one is asleep), sleep disturbances, use of sleeping medication, and daytime dysfunction.

TABLE 1

| | No CBD | | | | | | With CBD | | | | | |
|---|---|---|---|---|---|---|---|---|---|---|---|---|
| | PSQI | | PSQI | | PSQI | | PSQI | | PSQI | | ΔPSQI | |
| | M0 | STDV | M1 | STDV | M2 | STDV | M3 | STDV | M4 | STDV | (%) | Sedation |
| Group A | 12.0 | 1.0 | | | | | 6.7 | 1.1 | 4.6 | 0.6 | 62% | Y |
| Group B | 12.3 | 0.6 | | | | | 11.3 | 1.5 | 9.3 | 2.1 | 24% | N |
| Group C | 12.6 | 0.6 | 12.3 | 1.1 | 11.6 | 1.5 | 10.0 | 1.0 | 9.0 | 1.7 | 29% | N |
| Group D | 11.3 | 0.6 | 11.3 | 1.5 | 11.0 | 0.0 | 9.7 | 2.1 | 9.3 | 1.1 | 18% | N |
| Group E | 11.6 | 1.1 | 11.6 | 1.1 | 11.3 | 0.6 | 9.3 | 0.6 | 8.0 | 1.0 | 31% | N |
| Group F | 12.3 | 1.1 | 11.6 | 1.1 | 10.6 | 0.6 | 6.0 | 1.0 | 3.6 | 0.6 | 71% | N |

(M = month):

Each item is weighted on a 0-3 interval scale. The global PSQI score was calculated by totaling the seven component scores, providing an overall score ranging from 0 to 21, where lower scores denote a healthier sleep quality. A higher number indicates more sleep-related concerns. A score of 5 or greater indicates a “poor sleeper”. The results are presented in table 2, herein below.

TABLE 2

| | No CBD | | | | | | With CBD | | | | | |
|---|---|---|---|---|---|---|---|---|---|---|---|---|
| | PSQI | | PSQI | | PSQI | | PSQI | | PSQI | | ΔPSQI | |
| | M0 | STDV | M1 | STDV | M2 | STDV | M3 | STDV | M4 | STDV | (%) | Sedation |
| Group A | 12.0 | 1.0 | | | | | 6.7 | 1.1 | 4.6 | 0.6 | 62% | Y |
| Group B | 12.3 | 0.6 | | | | | 11.3 | 1.5 | 9.3 | 2.1 | 24% | N |
| Group C | 12.6 | 0.6 | 12.3 | 1.1 | 11.6 | 1.5 | 10.0 | 1.0 | 9.0 | 1.7 | 29% | N |
| Group D | 11.3 | 0.6 | 11.3 | 1.5 | 11.0 | 0.0 | 9.7 | 2.1 | 9.3 | 1.1 | 18% | N |
| Group E | 11.6 | 1.1 | 11.6 | 1.1 | 11.3 | 0.6 | 9.3 | 0.6 | 8.0 | 1.0 | 31% | N |
| Group F | 12.3 | 1.1 | 11.6 | 1.1 | 10.6 | 0.6 | 6.0 | 1.0 | 3.6 | 0.6 | 71% | N |

| Daily | CBD | EPA + DHA | DHA | EPA | Polar-EPA |
|---|---|---|---|---|---|
| Dosage | (mg) | (mg) | (mg) | (mg) | (mg) |
| Group A | 200 (High) | | | | |
| Group B | 50 (Low) | | | | |
| Group C | 50 (Low) | 300 (EPA) | | | |
| | | 200 (DHA) | | | |
| Group D | 50 (Low) | | 500 | | |
| Group E | 50 (Low) | | | 500 | |
| Group F | 50 (Low) | | | | 500 |

Omega-3 supplements, on a stand-alone basis, show a mild improvement in sleep quality. High-dose CBD was more efficient that low-dose CBD in improving PSQI score, but had a sedation effect. Results show a great difference in the combined effect between various Omega-3s and low-dosage CBD. While all Omega-3 oils+low-dose CBD showed higher efficacy compared with low-dose CBD on a stand-alone basis, only Nannochloropsis oil, which contains polar-lipid (glycolipid and phospholipid) EPA, was found to create a synergetic effect. The PSQI improvement after 2 months was about 3× higher than administrating low-dose CBD only and over 2× higher than fish oil+CBD. The Nannochloropsis oil+low dose CBD created a similar PSQI improvement as high-dose CBD, but without the sedation effect. On the other hand, DHA Omega-3, from Schizochytrium Oil, did not create any effect at all. Group F, months 1-2, indicate the improvement of PSQI score with Nannochloropsis (EPA only) on a stand-alone basis (~15%). Group B, months 3-4, indicate the improvement of PSQI score with low-dose CBD on a stand-alone basis (~18%). Group F, months 3-4, indicate the synergistic effect (64% improvement), indicating that the improvement is not a mere aggregation of features, but a product of a synergistic effect. This is the only group were the PSQI score fell under the “poor sleep” threshold (<5).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of treating a disease or disorder selected from sleep disorder, insomnia, depression, anxiety, attention-deficit disorder, weight disorder including obesity or anorexia, and any combinations thereof in a subject in need thereof; said method comprising administering a composition comprising:
Nannochloropsis oil comprising at least one poly-unsaturated fatty acid (PUFA) moiety; and
CBD or a derivative thereof.

2. The method of claim 1, wherein the at least one PUFA moiety is a glycolipid.

3. The method of claim 1, wherein the at least one PUFA moiety comprises at least one of moieties of omega-3, omega-6, omega 7, omega-9, conjugated unsaturated fatty acids and any combinations thereof.

4. The method of claim 1, wherein the at least one PUFA moiety comprises an omega-3 fatty acid moiety that comprises at least one of: Hexadecatrienoic acid (HTA), a-Linolenic acid (ALA), Stearidonic acid (SDA), Eicosatrienoic acid (ETE), Eicosatetraenoic acid (ETA), Eicosapentaenoic acid (EPA), Heneicosapentaenoic acid (HPA), Docosapentaenoic acid (DPA) Clupanodonic acid, Docosahexaenoic acid (DHA), Tetracosapentaenoic acid, Tetracosahexaenoic acid (Nisinic acid) and any combinations thereof.

5. The method of claim 1, wherein the at least one PUFA moiety comprises an omega-6 fatty acid that comprises at least one of: Linoleic acid (LA), Gamma-linolenic acid (GLA), Eicosadienoic acid, Dihomo-gamma-linolenic acid (DGLA), Arachidonic acid (AA), Docosadienoic acid, Adrenic acid (AdA), Docosapentaenoic acid (Osbond acid), Tetracosatetraenoic acid, Tetracosapentaenoic acid, and any combinations thereof.

6. The method of claim 1, wherein the at least one PUFA moiety comprises an omega-9 fatty acid that comprises at least one of: Oleic acid, Eicosenoic acid, Mead acid, Erucic acid, Nervonic acid, and any combinations thereof.

7. The method of claim 1, wherein the at least one PUFA moiety comprises at least one moieties of: Rumenic acid, a-Calendic acid, β-Calendic acid, Jacaric acid, a-Eleostearic acid, β-Eleostearic acid, Catalpic acid, Punicic acid, Rumelenic acid, a-Parinaric acid, β-Parinaric acid, Bosseopentaenoic acid, Pinolenic acid, Podocarpic acid and any combinations thereof.

8. The method of claim 1, wherein the at least one PUFA moiety comprises a moiety of DHA, EPA and any combinations thereof.

9. The method of claim 1, wherein the Nannochloropsis oil forms at least 20% wt of the composition.

10. The method of claim 1, wherein the CBD forms at least 10% wt of the composition.

11. The method of claim 1, wherein a ratio between the Nannochloropsis oil and the CBD is 1:1.

12. The method of claim 1, wherein the composition is an oily solution, an oil-in-water solution or a dry powder.
